(19) United States
(12) Patent Application Publication
(10) Pub. No.: US 2018/0087093 A1
(43) Pub. Date: Mar. 29, 2018

(54) PROBE FOR DETECTING DRUG RESISTANT GRAM-POSITIVE PATHOGENS, PROBE SET AND METHOD FOR DETECTING DRUG RESISTENT GRAM-POSITIVE PATHOGENS USING THEM

(71) Applicant: CATHOLIC UNIVERSITY INDUSTRY ACADEMIC COOPERATION FOUNDATION, Seoul (KR)

(72) Inventors: Yeun Jun CHUNG, Gyeonggido (KR); Dong Gun LEE, Seoul (KR); Chul Min PARK, Seoul (KR); Bo Ram CHUNG, Seoul (KR)

(21) Appl. No.: 15/617,135

(22) Filed: Jun. 8, 2017

(30) Foreign Application Priority Data
Sep. 23, 2016 (KR) 10-2016-0122126

(51) Int. Cl. C12Q 1/68

(52) U.S. Cl. CPC C12Q 1/689; C12Q 2600/156; C12Q 1/6818

(57) ABSTRACT

The present invention relates to a probe for detecting drug-resistant gram-positive pathogens, a probe set, and a method of detecting drug-resistant gram-positive pathogens using them. The probe for detecting drug-resistant gram-positive pathogens, the probe set, and the method for detecting drug-resistant gram-positive pathogens using them according to the present invention can contribute not only to shortening a detection time, compared to a conventional detection time, by optimizing a hybridization time, but also to increasing the sensitivity of detection by pre-amplification of the specific sequence of drug-resistant gram-positive pathogens, and thereby multiple detections of drug-resistant gram-positive pathogens can be accurately and efficiently performed.

### CROSS-REFERENCE TO RELATED APPLICATION

This application claims priority to and the benefit of Korean Patent Application No. 10-2016-0122126, filed on Sep. 23, 2016, the disclosure of which is incorporated herein by reference in its entirety.

### BACKGROUND

### 1. Field of the Invention

The present invention relates to a probe for detecting drug-resistant gram-positive pathogens, a probe set, and a method of detecting drug-resistant gram-positive pathogens using them.

### 2. Discussion of Related Art

Sepsis is a disease which is the leading cause of death in an intensive care unit (ICU). According to U.S statistics, 750,000 people are diagnosed with sepsis every year and, among them, 210,000 people die. Also, annually, sepsis is a disease which is the 10^{th} (6% of total deaths) leading cause of death. Currently, the incidence thereof has tripled since the early 1970s and is increasing by about 1.5% every year. As high-risk treatment and treatment use increase according to population aging, it is expected that deaths caused by sepsis will reach 1.1 million people per year in 2020. In Korea, the number of deaths caused by sepsis is about 8,000 per year and has been continuously increasing since 2008.

A gram-positive pathogen (gram-positive bacteria) is the most common organism responsible for sepsis. Examples of a major drug-resistant gram-positive pathogen include Staphylococcus aureus, Enterococcus faecium, Streptococcus pneumoniae and the like. Among these, methicillin-resistant Staphylococcus aureus, vancomycin-resistant Enterococcus francium and the like, all of which are resistant to drugs, are included. The drug-resistant gram-positive pathogens are important in treating infectious diseases caused by drug resistance.

Early detection of drug-resistant bacterial pathogens from the blood of sepsis patients is important in improving clinical results. To achieve this, the rapid and sensitive detection of drug-resistant gram-positive pathogens is important. However, existing cell culture-based methods take a relatively long time to identify drug-resistant pathogens and thus have a disadvantage in which the overall detection time is longer. To replace these methods, there are two molecular biological diagnostic methods using polymerase chain reaction (PCR). One is multiplex PCR and the other is real-time PCR, both of which have advantages and disadvantages. Multiplex PCR allows the detection of a plurality of pathogens from the same sample, and thus a plurality of candidate pathogens can be effectively screened. However, multiplex detection can result in errors in the result analysis because a variety of primer sets are attached to genomic DNA to form non-specific hybridization and thus other products other than amplified products to be detected are amplified. Therefore, multiplex PCR has less specificity than real-time PCR. Real-time PCR provides relatively accurate results, but is limited in multiplex detection capability.

Multiplex ligation-dependent probe amplification (MLPA) is a method for detecting only a specific DNA base sequence and is a molecular biological diagnostic method having high specificity and capable of multiplex detection of a variant through a single experiment.

In MLPA, two probes including a sequence that hybridizes to a target sample and a primer sequence are used. When each of the two probes is hybridized to a target gene and two hybridized probes are ligated to form a probe assembly, it is only possible to perform an amplification reaction using the probe assembly as a template by a primer included in each of two probes in the subsequent amplification reaction. Therefore, since the extent of amplification and a detection amount thereon are varied depending on the amount of the probe assembly obtained by hybridizing the probes to target genes and ligating hybridized probes, specific genes can be detected. In the case of existing MLPA, capillary electrophoresis (CE) is used for detection, and thus there is a stuffer sequence that is designed to have a difference in length in a process of designing a probe for detection. A stuffer sequence refers to a sequence that is designed to simply have a difference in length and is a base sequence that does not bind to hybridization sequences of other probes, stuffer sequences of other probes or other base sequences of DNA. However, the stuffer sequence, as described above, has a disadvantage in which it is difficult to design the stuffer sequence itself because the stuffer sequence should not interact with other base sequences. In addition, even when the base sequence for the stuffer sequence is designed, a difference in length between probes increases due to a stuffer sequence having a maximum length of about 400 nt, and thus amplification efficiency of the entire product is varied, which leads to difficulty in accurate detection.

In order to solve these problems, stuffer-free MLPA without a stuffer sequence is used in the present invention and thus it is possible to easily design probes and allow accurate detection due to constant amplification efficiency.

In order to detect a probe for stuffer-free MLPA which does not have a difference in length, in the present invention, capillary electrophoresis-single strand conformation polymorphism (CE-SSCP) is applied to detect a probe using a difference in not length but base sequence. A process of detecting a strain by the stuffer-free MLPA-CE-SSCP method is schematically illustrated in FIG. 1. As shown in FIG. 1, the MLPA method includes denaturing a sample and hybridizing two probes to a target gene, ligating the hybridized two probes, performing PCR amplification of the ligated probes, and detecting an amplified product.

However, a method of detecting drug-resistant gram-positive pathogens based on the stuffer-free MLPA-CE-SSCP method has not yet been developed. Therefore, it is necessary to develop a method of rapidly and accurately detecting drug-resistant gram-positive pathogens based on the stuffer-free MLPA-CE-SSCP method.

### SUMMARY OF THE INVENTION

The present invention is designed to solve the problems of the prior art as described above, and it is an object of the present invention to provide a probe for detecting stuffer-free drug-resistant gram-positive pathogens without a stuffer sequence for detecting drug-resistant gram-positive pathogens by multiplex ligation-dependent probe amplification (MLPA), a probe set, and a method of detecting drug-resistant gram-positive pathogens.

In addition, it is an object of the present invention to shorten a detection time by optimizing a hybridization time and to increase the sensitivity of detection by performing pre-amplification of the specific sequence of drug-resistant gram-positive pathogens using the probe for detecting drug-resistant gram-positive pathogens and a probe set.

In order to solve the above problems, the present invention provides a probe for detecting gram-positive pathogens associated with sepsis, which is composed of:

a left probe oligonucleotide (LPO) including a forward primer binding site and a left hybridization sequence (LHS) region that specifically hybridizes to a first region of a target sequence; and

a right probe oligonucleotide (RPO) including a reverse primer binding site and a right hybridization sequence (RHS) region that specifically hybridizes to a second region continuously connected with the first region of the target sequence,

wherein the LHS and the RHS are any one selected from the group consisting of a first probe of SEQ ID NO: 1 and SEQ ID NO: 2, a second probe of SEQ ID NO: 3 and SEQ ID NO: 4, a third probe of SEQ ID NO: 5 and SEQ ID NO: 6, a fourth probe of SEQ ID NO: 7 and SEQ ID NO: 8, and a fifth probe of SEQ ID NO: 9 and SEQ ID NO: 10.

In the present invention, the gram-positive pathogens may be any one or more selected from the group consisting of Staphylococcus aureus, methicillin-resistant Staphylococcus aureus, Enterococcus faecium, vancomycin-resistant Enterococcus faecium and Streptococcus pneumoniae.

In the present invention, the first probe of SEQ ID NO: 1 and SEQ ID NO: 2 may specifically hybridize to SEQ ID NO: 11 of a nuc gene of Staphylococcus aureus,

the second probe of SEQ ID NO: 3 and SEQ ID NO: 4 may specifically hybridize to SEQ ID NO: 12 of a mecA gene of methicillin-resistant Staphylococcus aureus,

the third probe of SEQ ID NO: 5 and SEQ ID NO: 6 may specifically hybridize to SEQ ID NO: 13 of a sodA gene of Enterococcus faecium,

the fourth probe of SEQ ID NO: 7 and SEQ ID NO: 8 may specifically hybridize to SEQ ID NO: 14 of a vanA gene of vancomycin-resistant Enterococcus faecium, and

the fifth probe of SEQ ID NO: 9 and SEQ ID NO: 10 may specifically hybridize to SEQ ID NO: 15 of a lytA gene of Streptococcus pneumoniae.

The present invention also provides a probe set for detecting gram-positive pathogens associated with sepsis, which includes two to five probes for detecting gram-positive pathogens associated with sepsis according to the present invention.

The present invention also provides a kit for detecting gram-positive pathogens associated with sepsis, which includes the probe for detecting gram-positive pathogens associated with sepsis according to the present invention or the probe set according to the present invention.

The present invention also provides a method of detecting gram-positive pathogens associated with sepsis, which includes the following steps:

(1) a step of performing pre-amplification of a sample;

(2) a step of bringing the pre-amplified sample into contact with the probe set for detecting gram-positive pathogens associated with sepsis according to the present invention to induce hybridization;

(3) a step of ligating the probes hybridized to the sample to form a probe assembly;

(4) a step of performing PCR amplification by using the formed probe assembly as a template;

(5) a step of isolating and detecting the amplified product; and

(6) a step of determining that, when the amplified product is isolated and detected, there are gram-positive pathogens associated with sepsis to which probes constituting the amplified probe assembly complementarily bind in the sample.

In the present invention, in the step (1) of performing pre-amplification of a sample, a first primer set to a fifth primer set may be used to amplify the first region of a target sequence of a strain in the sample and the second region continuously connected with the first region.

In the present invention, the first primer set of SEQ ID NO: 16 and SEQ ID NO: 17 may amplify a first region and a second region of the nuc gene of Staphylococcus aureus,

the second primer set of SEQ ID NO: 18 and SEQ ID NO: 19 may amplify a first region and a second region of the mecA gene of methicillin-resistant Staphylococcus aureus,

the third primer set of SEQ ID NO: 20 and SEQ ID NO: 21 may amplify a first region and a second region of the sodA gene of Enterococcus faecium,

the fourth primer set of SEQ ID NO: 22 and SEQ ID NO: 23 may amplify a first region and a second region of the vanA gene of vancomycin-resistant Enterococcus faecium, and

the fifth primer set of SEQ ID NO: 24 and SEQ ID NO: 25 may amplify a first region and a second region of the lytA gene of Streptococcus pneumoniae.

In the present invention, in the step (2), the first region of the target sequence of the sample may be hybridized to the LHS of the LPO probe, and the second region of the target sequence of the sample may be hybridized to the RHS of the RPO probe.

In the present invention, the hybridization in the step (2) may be performed for 4 to 20 hours, preferably 4 to 16 hours, more preferably 8 to 16 hours, and most preferably 8 hours.

In the present invention, in the step (3), the end of the LHS of the LPO hybridized to the first region of the sample and the end of the RHS of the RPO hybridized to the second region of the sample may be ligated to form the probe assembly composed of the forward primer binding site-LHS-RHS-the reverse primer binding site.

In the present invention, in the step (4), a primer set complementarily binding to the forward primer binding site included in the LPO and the reverse primer binding site included in the RPO, which constitute the probe assembly, may be used to perform PCR amplification of the formed probe assembly.

In the present invention, in the step (5), an extent at which the probe assembly as a template is PCR-amplified may be isolated and detected using capillary electrophoresis-single strand conformation polymorphism (CE-SSCP).

In the present invention, in the step (2), the presence of a plurality of gram-positive pathogens may be detected at one time using the probe set for detecting gram-positive pathogens associated with sepsis according to the present invention.

The present invention also provides a method of providing information for the diagnosis of sepsis by using any one method of the above methods.

The present invention provides a probe for detecting drug-resistant gram-positive pathogens associated with sepsis, which is composed of a left probe oligonucleotide (LPO) including a forward primer binding site and a left hybridization sequence (LHS) region that specifically hybridizes to a first region of a target sequence; and

a right probe oligonucleotide (RPO) including a reverse primer binding site and a right hybridization sequence (RHS) region that specifically hybridizes to a second region continuously connected with the first region of the target sequence,

wherein the LHS and the RHS are any one selected from the group consisting of a second probe of SEQ ID NO: 3 and SEQ ID NO: 4 and a fourth probe of SEQ ID NO: 7 and SEQ ID NO: 8.

In the present invention, the drug-resistant gram-positive pathogens may be any one or more selected from the group consisting of methicillin-resistant Staphylococcus aureus and vancomycin-resistant Enterococcus faecium.

In the present invention, the second probe of SEQ ID NO: 3 and SEQ ID NO: 4 may specifically hybridize to SEQ ID NO: 12 of a mecA gene of methicillin-resistant Staphylococcus aureus, and

the fourth probe of SEQ ID NO: 7 and SEQ ID NO: 8 may specifically hybridize to SEQ ID NO: 14 of a vanA gene of vancomycin-resistant Enterococcus faecium.

The present invention also provides a probe set for detecting drug-resistant gram-positive pathogens associated with sepsis, which includes two probes for detecting drug-resistant gram-positive pathogens associated with sepsis according to the present invention.

The present invention also provides a kit for detecting drug-resistant gram-positive pathogens associated with sepsis, which includes the probe for detecting drug-resistant gram-positive pathogens associated with sepsis according to the present invention or the probe set according to the present invention.

The present invention also provides a method of detecting drug-resistant gram-positive pathogens associated with sepsis, which includes the following steps:

(1) a step of performing pre-amplification of a sample;

(2) a step of bringing the pre-amplified sample into contact with the probe set for detecting drug-resistant gram-positive pathogens associated with sepsis according to the present invention to induce hybridization;

(3) a step of ligating the probes hybridized to the sample to form a probe assembly;

(4) a step of performing PCR amplification by using the formed probe assembly as a template;

(5) a step of isolating and detecting the amplified product; and

(6) a step of determining that, when the amplified product is isolated and detected, there are drug-resistant gram-positive pathogens associated with sepsis to which probes constituting the amplified probe assembly complementarily bind in the sample.

In the present invention, in the step (1) of performing pre-amplification of a sample, a second primer set and a fourth primer set may be used to amplify the first region of a target sequence of a strain in the sample and the second region continuously connected with the first region.

In the present invention, the second primer set of SEQ ID NO: 18 and SEQ ID NO: 19 may amplify a first region and a second region of a mecA gene of methicillin-resistant Staphylococcus aureus, and

the fourth primer set of SEQ ID NO: 22 and SEQ ID NO: 23 may amplify a first region and a second region of a vanA gene of vancomycin-resistant Enterococcus faecium.

In the present invention, in the step (2), the first region of the target sequence of the sample may hybridizes to the LHS of the LPO probe, and the second region of the target sequence of the sample may hybridizes to the RHS of the RPO probe.

In the present invention, the hybridization in the step (2) may be performed for 4 to 20 hours, preferably 4 to 16 hours, more preferably 8 to 16 hours, and most preferably 8 hours.

In the present invention, in the step (3), the end of the LHS of the LPO hybridized to the first region of the sample and the end of the RHS of the RPO hybridized to the second region of the sample may be ligated to form the probe assembly composed of the forward primer binding site-LHS-RHS-the reverse primer binding site.

In the present invention, in the step (4), a primer set complementarily binding to the forward primer binding site included in the LPO and the reverse primer binding site included in the RPO, which constitute the probe assembly, may be used to perform PCR amplification of the probe assembly.

In the present invention, in the step (5), an extent at which the probe assembly as a template is PCR-amplified may be isolated and detected using capillary electrophoresis-single strand conformation polymorphism (CE-SSCP).

In the present invention, in the step (2), the presence of a plurality of drug-resistant gram-positive pathogens may be detected at one time using the probe set for detecting drug-resistant gram-positive pathogens associated with sepsis according to the present invention.

The present invention also provides a method of providing information for the diagnosis of drug-resistant sepsis by using any one method of the above methods.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a flowchart schematically illustrating a stuffer-free multiplex ligation-dependent probe amplification (MLPA) method according to the present invention;

FIG. 2 is a diagram illustrating the structure of a probe for detecting drug-resistant gram-positive pathogens without a stuffer sequence according to the present invention;

FIG. 3 is a diagram illustrating a result obtained by performing MLPA using a probe for detecting drug-resistant gram-positive pathogens according to one embodiment of the present invention and analysis using capillary electrophoresis-single strand conformation polymorphism (CE-SSCP);

FIG. 4 is a diagram illustrating a result obtained by performing MLPA for 60 pathogens isolated from a clinic using a probe for detecting drug-resistant gram-positive pathogens according to the present invention and detection using CE-SSCP according to one example of the present invention;

FIG. 5 is a diagram illustrating a result obtained by diluting genomic DNA isolated from strains prepared in Example 3 to concentrations of 100 pg, 10 pg, 1 pg, 100 fg and 10 fg to perform pre-amplification using a primer for detecting drug-resistant gram-positive pathogens and then analysis using MLPA and CE-SSCP according to one example of the present invention; and

FIG. 6 is a diagram illustrating a result obtained by performing pre-amplification using a primer for detecting drug-resistant gram-positive pathogens and then analysis using MLPA and CE-SSCP according to one example of the present invention.

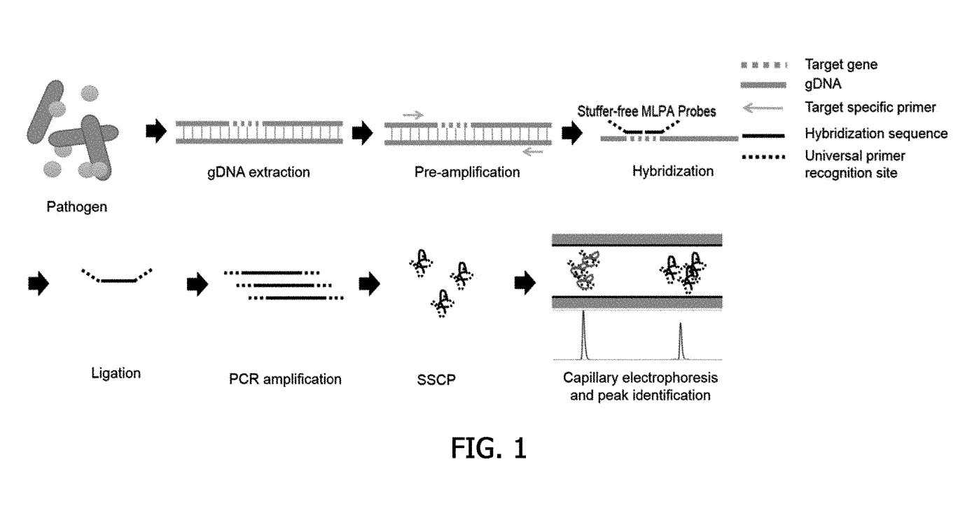

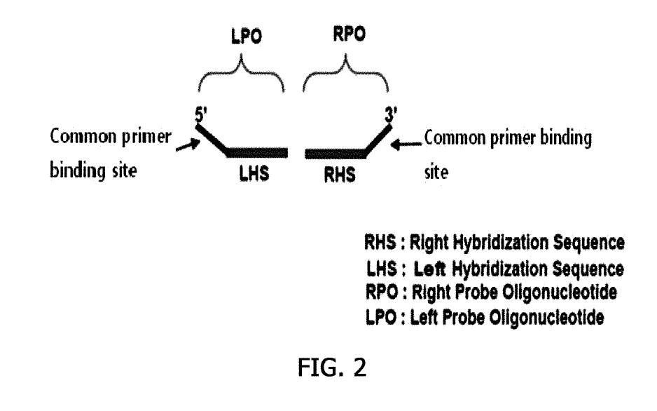

### DETAILED DESCRIPTION OF EXEMPLARY EMBODIMENTS

Hereinafter, the present invention will be described in more detail with reference to examples of the present invention. However, the present invention is not limited by the following examples.

### Example

### Example 1. Determination of Sequence of Drug-Resistant Gram-Positive Pathogen Strain to be Detected

Through a document search for a total of 5 strains widely known as drug-resistant gram-positive pathogen strains, such as Staphylococcus aureus, methicillin-resistant staphylococcus aureus (MRSA), Enterococcus faecium, vancomycin-resistant Enterococcus faecium (VREfm) and Streptococcus pneumoniae, species-specific genes or sequences used to detect each strain gene were selected.

After securing the genes and the base sequences used to detect each strain gene, the genes and sequences were compared to a DNA sequence database of each strain using the Basic Local Alignment Search Tool (BLAST) developed by the National Center for Biotechnology Information (NCBI), and then specific base sequences were determined.

The sequence region commonly included in each strain was determined as a region to which a probe specifically hybridizes. The results are shown in the following Table 1. The sequence region of each strain was composed of a first region of a target sequence to which a left hybridization sequence (LHS) specifically hybridizes and a second region of the target sequence continuously connected with the first region and to which a right hybridization sequence (RHS) specifically hybridizes in combination.

TABLE 1

| | | SEQ |
|---|---|---|
| | | ID |
| Species | Hybridization sequence | NO: |
| Staphylococcus | CAACATCACGAAATTCCTTACATGACCTCGGTTTAGT | 11 |
| aureus | TCACAGAAGCCGTGTTCTCATC | |
| MRSA | GGTCAATAATGTCCCAGCTAGAATGCAGTATGAAAA | 12 |
| | AATAACGGCTCACAGCATGGAACAACT | |
| Enterococcus | CCTGCTTCGACCTTCAAAATGCTTAATGCTTTGATCG | 13 |
| faecium | GCCTTGAGCACCATAAGGCAAC | |
| VREfm | GGCTCAGGTACTGCTATCCACCCTCAAACAGGTGAA | 14 |
| | TTATTAGCACTTGTAAGCACACC | |
| Streptococcus | CATCCTAAAAAAGGTGTAGAGAAATATGGTCCTGAA | 15 |
| pneumoniae | GCAAGTGCATTTACGAAAAAGATGGT | |

### Example 2. Design of Probe for Detecting Strain

Based on the specific sequences determined in Example 1, a probe for detecting drug-resistant gram-positive pathogens was designed. The specificity of the selected genes or sequences was identified using BLAST, and then a left hybridization sequence (LHS) and a right hybridization sequence (RHS) were designed so that a ligation site was included in the identified genes or sequences.

In this case, each of the LHS and the RHS was designed so as to have a sequence length ranging from 21 nt to 50 nt, a Tm ranging from 70 to 80° C. and a GC content ranging from 35 to 65%. Specificities of the LHS and RHS thus designed were identified using FASTA or BLAST.

The homology of all the sequences of the LHS and RHS thus identified was compared so as to exhibit an alignment score of 50 or less, and common primer binding sequences were added to the LHS and RHS that were identified to exhibit an alignment score of 50 or less to form a left hybridization oligonucleotide (LHO) and a right hybridization oligonucleotide (RHO). Afterward, probes for detecting drug-resistant gram-positive pathogens were completed in such a way that each of the LHO and the RHO has a DG value of 0 or more. The probes for detecting drug-resistant gram-positive pathogens thus completed are shown in the following Table 2.

TABLE 2

| | | | | SEQ |
|---|---|---|---|---|
| | | | | ID |
| Species | Gene | Position | Probe sequence | NO: |
| Staphylococcus | nuc | LHS | CAACATCACGAAATTCCTTACAT | 1 |
| aureus | | | GACCTCG | |
| | | RHS | GTTTAGTTCACAGAAGCCGTGTT | 2 |
| | | | CTCATC | |
| MRSA | mecA | LHS | GGTCAATAATGTCCCAGCTAGAA | 3 |
| | | | TGCAGTATGAAA | |
| | | RHS | AAATAACGGCTCACAGCATGGA | 4 |
| | | | ACAACT | |
| Enterococcus | sodA | LHS | CCTGCTTCGACCTTCAAAATGCT | 5 |
| faecium | | | TAATGCTTTG | |
| | | RHS | ATCGGCCTTGAGCACCATAAGGC | 6 |
| | | | AAC | |
| VREfm | vanA | LHS | GGCTCAGGTACTGCTATCCACCC | 7 |
| | | | TCAA | |
| | | RHS | ACAGGTGAATTATTAGCACTTGT | 8 |
| | | | AAGCACACC | |
| Streptococcus | lytA | LHS | CATCCTAAAAAAGGTGTAGAGA | 9 |
| pneumoniae | | | AATATGGTCCT | |
| | | RHS | GAAGCAAGTGCATTTACGAAA | 10 |
| | | | AAGATGGT | |

### Example 3. Detection Experiment Using Probe for Detecting Drug-Resistant Gram-Positive Pathogens

3-1. Preparation of DNA Sample of Strain

Staphylococcus aureus, methicillin-resistant staphylococcus aureus (MRSA), Enterococcus faecium, vancomycin-resistant Enterococcus faecium (VREfm) and Streptococcus pneumoniae which were identified in the American Type Culture Collection (ATCC), Korean Collection of Type Culture (KCTC), Statens Serum Institut (SSI) and National Culture Collection for Pathogens (NCCP) were selected as reference strains in the method of detecting drug-resistant gram-positive pathogens.

60 gram-positive pathogens isolated from a clinic and collected in the Korean Centers for Disease Control and Prevention (KCDC) and Seoul St. Mary's Hospital were used to identify the specificity of the method of detecting drug-resistant gram-positive pathogens.

4 strains except S. pneumoniae were cultured in nutrient broth, S. pneumoniae was cultured in trypticase soy agar with 5% sheep blood, and then genomic DNA was extracted using the GeneAll Cell SV kit (commercially available from GeneAll Biotechnology Co., Ltd., Seoul, Korea).

3-2. Performance of MLPA

MLPA was performed using a Lig-5a kit commercially available from MRC-Holland (www.MLPA.com).

5 μL of each of the genomic DNA samples of the strains was denatured at 95° C. for 5 minutes, 1.5 μL of a MLPA buffer (commercially available from MRC Holland, the Netherlands) and 1.5 μL of a probe for detecting drug-resistant gram-positive pathogens were then added thereto, and the resulting substance was reacted at 60° C. for 16 hours to hybridize a target sequence of the sample and two probes (LHO and RHO).

After the hybridization for 16 hours, a reaction temperature of the sample was decreased to 54° C., and then 1 μL of Ligase-65, 3 μL of each of Ligase-65 buffer A and B and 25 μL of tertiary distilled water were added to induce a reaction at 54° C. for 15 minutes. Two probes (LHO and RHO) thus hybridized were ligated to form a probe assembly. Afterward, an enzyme was inactivated at 98° C. for 5 minutes.

PCR amplification of the probe assembly thus formed was performed by binding common primers to primer binding sites included in the two probes (LHO and RHO). As the common primers, GGGTTCCCTAAGGGTTGGA was used as a forward primer and GTGCCAGCAAGATCCAATCTAGA was used as a reverse primer.

4 μL of the probe assembly thus formed and 20 μL of a mixed solution which was adjusted by adding tertiary distilled water to a 10 mM forward primer and a 10 mM reverse primer as common primers were reacted using a pfu-PCR premix (commercially available from Bioneer Corporation, Daejeon, Korea) 35 times at 95° C. for 30 seconds, at 60° C. for 30 seconds and at 72° C. for 30 seconds to amplify the ligated probe assembly.

3-3. Detection of Amplified Product Using CE-SSCP

A stuffer-free MLPA product thus obtained was diluted with tertiary distilled water and then mixed with 1 μL of the sample, 0.3 μL of a size standard (commercially available from Applied Biosystems, Inc., Foster City, Calif.) and 8.7 μL of deionized formamide to induce a reaction at 95° C. for 5 minutes and at 4° C. for 4 minutes. The CE-SSCP result for this sample was confirmed using the 3130x1 Genetic Analyzer (commercially available from Applied Biosystems, Inc.).

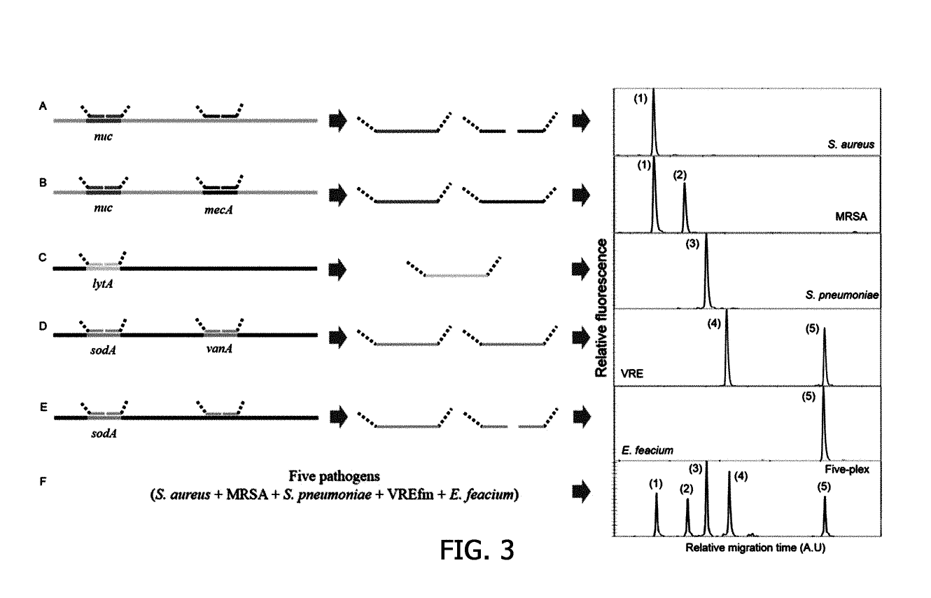

FIG. 3 is a diagram illustrating results obtained by performing MLPA individually using the probe for detecting drug-resistant gram-positive pathogens and analyzing the amplified probe assembly using CE-SSCP in Example 3 of the present invention.

Referring to FIG. 3, it can be seen that the probe for detecting drug-resistant gram-positive pathogens according to the present invention exhibits a specific peak with respect to each gene. In addition, it can be seen that, even when a plurality of probes were used, a corresponding strain can be independently detected and analyzed at the same time.

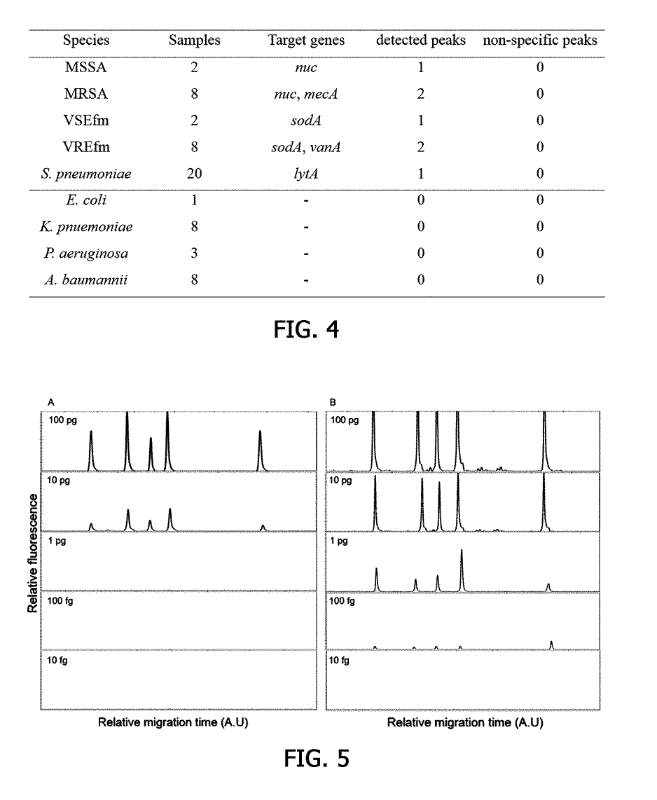

FIG. 4 illustrates results obtained by identifying the specificity of the method of detecting drug-resistant gram-positive pathogens using 60 pathogens isolated from a clinic and collected in the Korean Centers for Disease Control and Prevention (KCDC) and Seoul St. Mary's Hospital. In FIG. 4, it was confirmed that 40 pathogens of 60 pathogens isolated from the clinic were gram-positive, in which 8 pathogens were MRSA, 2 pathogens were MSSA, 8 pathogens were VREfm, 2 pathogens were VSEfm and 20 pathogens were S. pneumoniae, and 20 pathogens were gram-negative, in which 1 pathogen was Escherichia coli, 8 pathogens were Klebsiella pneumoniae, 3 pathogens were Pseudomonas aeruginosa and 8 pathogens were Acinetobacter baumannii.

### Example 4. Detection Experiment with Addition of Pre-Amplification Process

4-1. Performance of Pre-Amplification

In order to pre-amplify a region including species-specific base sequences as described above, gene-specific primers were designed by using primer3 (http://frodo.wi.mit.edu/), results of which are shown in Table 3.

TABLE 3

| | | | | SEQ |
|---|---|---|---|---|
| | | | | ID |
| Species | Gene | Primer | Primer sequence | NO: |
| Staphylococcus | nuc | Forward | TACGGCAACCTCTTTCCATC | 16 |
| aureus | | Reverse | CATGCCCTTCTCCCTTTGTA | 17 |
| MRSA | mecA | Forward | AGCGGTAAACGATTTGTTGG | 18 |
| | | Reverse | GATAGACTTGGCGCCCATAA | 19 |
| Enterococcus | sodA | Forward | AGCTCAGCAAATGCATCACA | 20 |
| faecium | | Reverse | AGCCAAGCCTTGACGAACTA | 21 |
| VREfm | vanA | Forward | GGCTATCGTGTCACAATCGTT | 22 |
| | | Reverse | TGGAACTTGTTGAGCAGAGG | 23 |
| Streptococcus | lytA | Forward | CAAATCACAGCGCTTCAAAA | 24 |
| pneumoniae | | Reverse | AACCAACACGCTTCACTTCC | 25 |

The samples isolated from the strains prepared in Example 3 and 20 μL of a mixed solution which was adjusted by adding tertiary distilled water to 5 μL of genomic DNA and 10 mM of each of the forward primer and the reverse primer synthesized in Table 3 were initially denatured at 98° C. for 10 seconds, denatured at 98° C. for 30 seconds, annealed at 53° C. for 30 seconds, and elongated at 72° C. for 60 seconds 30 times using a high-fidelity DNA polymerase kit (commercially available from New England Biolabs) and DNA using a high-fidelity DNA polymerase kit (commercially available from New England Biolabs, Ipswich) to pre-amplify regions including probe-specific base sequences.

4-2. Performance of Pre-Amplification of Genomic DNA Depending on Diluted Concentration

In order to measure the sensitivity of a probe for detecting drug-resistant gram-positive pathogens, genomic DNA isolated from the strains prepared in Example 3 was diluted to a concentration of 1 ng to 1 fg to pre-amplify regions including probe-specific base sequences in the same manner as Example 4-1.

4-3. Performance of MLPA

MLPA was performed using a Lig-5a kit commercially available from MRC-Holland (www.MLPA.com).

5 μL of the pre-amplified product sample was denatured at 95° C. for 5 minutes, and then 1.5 μL of a mixed solution of 1.5 μL of a MLPA buffer (commercially available from MRC Holland, The Netherlands) and the probe for detecting drug-resistant gram-positive pathogens prepared in Example 3 was added thereto. Afterward, a target sequence of the sample and two probes (LHO and RHO) were hybridized at 60° C. for varying times (1 hour, 2 hours, 4 hours, 8 hours and 16 hours).

After the hybridization, a reaction temperature of the sample was decreased to 54° C., and then 1 μL of Ligase-65, 3 μL of each of Ligase-65 buffer A and B and 25 μL of tertiary distilled water were added to induce a reaction at 54° C. for 15 minutes. Two probes (LHO, RHO) thus hybridized were ligated to form a probe assembly. Afterward, an enzyme was inactivated at 98° C. for 5 minutes.

PCR amplification of the probe assembly thus formed was performed by binding common primers to primer binding sites included in the two probes (LHO and RHO). As the common primers, GGGTTCCCTAAGGGTTGGA was used as a forward primer, and GTGCCAGCAAGATCCAATCTAGA was used as a reverse primer.

4 μL of the probe assembly thus formed and 20 ml of a mixed solution which was adjusted by adding tertiary distilled water to a 10 mM forward primer and a 10 mM reverse primer as common primers were reacted using a pfu-PCR premix (commercially available from Bioneer Corporation, Daejeon, Korea) 35 times at 95° C. for 30 seconds, at 60° C. for 30 seconds and at 72° C. for 30 seconds to amplify the ligated probe assembly.

4-4. Detection of Amplified Product Using CE-SSCP

The amplified stuffer-free MLPA product obtained in Examples 4-2 and 4-3 was diluted with tertiary distilled water and then mixed with 1 μL of the sample, 0.3 μL of a size standard (commercially available from Applied Biosystems, Inc., Foster City, Calif.) and 8.7 μL of deionized formamide to induce a reaction at 95° C. for 5 minutes and at 4° C. for 4 minutes. The CE-SSCP result for this sample was confirmed using the 3130x1 Genetic Analyzer (commercially available from Applied Biosystems, Inc.).

FIG. 5 is an analysis result obtained by diluting genomic DNA isolated from the strains prepared in Example 3 to concentrations of 100 pg, 10 pg, 1 pg, 100 fg and 10 fg to perform pre-amplification using the primers for detecting drug-resistant gram-positive pathogens in Table 3 and performing stuffer-free MLPA-CE-SSCP for an amplified region. As shown in FIG. 5, it can be seen that, even when genomic DNA isolated from the strains prepared in Example 3 was diluted to concentrations of 100 pg, 10 pg, 1 pg, 100 fg and 10 fg to perform pre-amplification and then stuffer-free MLPA, strains were detected at a concentration of genomic DNA of 10 pg as shown in FIG. 3 in Example 3.

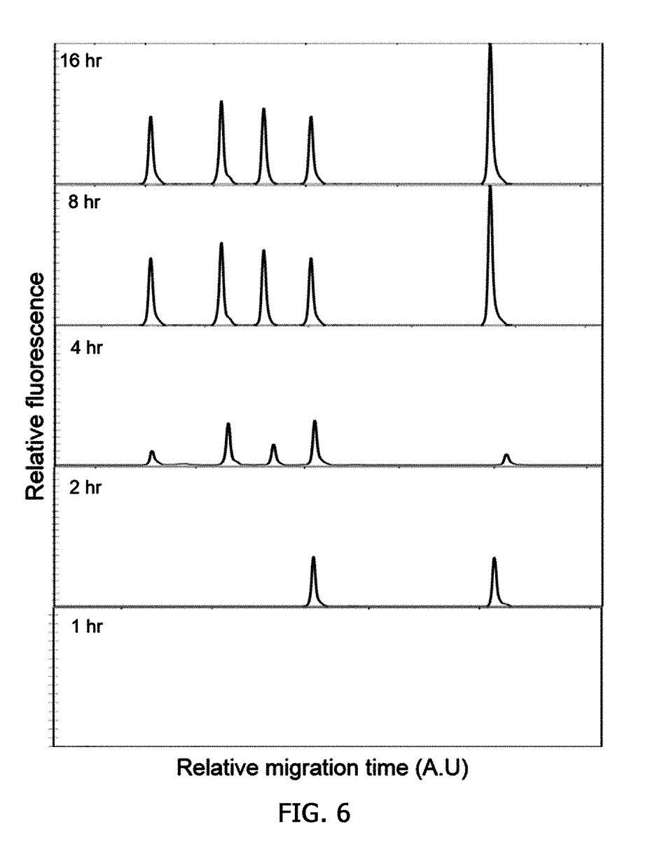

FIG. 6 is an analysis result obtained by performing pre-amplification using the primers for detecting drug-resistant gram-positive pathogens in Table 3 and performing stuffer-free MLPA-CE-SSCP for an amplified region. As shown in FIG. 6, it can be seen that, even when pre-amplification was performed according to the present invention and then stuffer-free MLPA was performed, five strains were simultaneously detected as shown in FIG. 3 in Example 3, and even when hybridization was performed for 4 hours, five strains also were simultaneously detected.

The probe for detecting drug-resistant gram-positive pathogens, the probe set, and the method for detecting drug-resistant gram-positive pathogens using them according to the present invention can contribute not only to shortening a detection time, compared to a conventional detection time, by optimizing a hybridization time, but also to increasing the sensitivity of detection by pre-amplification of the specific sequence of drug-resistant gram-positive pathogens, and thereby multiple detections of drug-resistant gram-positive pathogens can be accurately and efficiently performed.

What is claimed is:

1. A probe for detecting gram-positive pathogens associated with sepsis, the probe comprising:
a left probe oligonucleotide (LPO) including a forward primer binding site and a left hybridization sequence (LHS) region that specifically hybridizes to a first region of a target sequence; and
a right probe oligonucleotide (RPO) including a reverse primer binding site and a right hybridization sequence (RHS) region that specifically hybridizes to a second region continuously connected with the first region of the target sequence,
wherein the LHS and the RHS are any one or more selected from the group consisting of a first probe of SEQ ID NO: 1 and SEQ ID NO: 2, a second probe of SEQ ID NO: 3 and SEQ ID NO: 4, a third probe of SEQ ID NO: 5 and SEQ ID NO: 6, a fourth probe of SEQ ID NO: 7 and SEQ ID NO: 8, and a fifth probe of SEQ ID NO: 9 and SEQ ID NO: 10.

2. The probe of claim 1, wherein the gram-positive pathogens are any one or more selected from the group consisting of Staphylococcus aureus, methicillin-resistant Staphylococcus aureus, Enterococcus faecium, vancomycin-resistant Enterococcus faecium and Streptococcus pneumoniae.

3. The probe of claim 1, wherein the first probe of SEQ ID NO: 1 and SEQ ID NO: 2 specifically hybridizes to SEQ ID NO: 11 of a nuc gene of Staphylococcus aureus,
the second probe of SEQ ID NO: 3 and SEQ ID NO: 4 specifically hybridizes to SEQ ID NO: 12 of a mecA gene of methicillin-resistant Staphylococcus aureus,
the third probe of SEQ ID NO: 5 and SEQ ID NO: 6 specifically hybridizes to SEQ ID NO: 13 of a sodA gene of Enterococcus faecium,
the fourth probe of SEQ ID NO: 7 and SEQ ID NO: 8 specifically hybridizes to SEQ ID NO: 14 of a vanA gene of vancomycin-resistant Enterococcus faecium, and
the fifth probe of SEQ ID NO: 9 and SEQ ID NO: 10 specifically hybridizes to SEQ ID NO: 15 of a lytA gene of Streptococcus pneumoniae.

4. A method of detecting gram-positive pathogens associated with sepsis, the method comprising:
(1) a step of performing pre-amplification of a sample;
(2) a step of bringing the pre-amplified sample into contact with the probe for detecting gram-positive pathogens associated with sepsis according to claim 1 to induce hybridization;
(3) a step of ligating the probes hybridized to the sample to form a probe assembly;
(4) a step of performing PCR amplification by using the formed probe assembly as a template;
(5) a step of isolating and detecting the amplified product; and
(6) a step of determining that, when the amplified product is isolated and detected, there are gram-positive pathogens associated with sepsis to which probes constituting the amplified probe assembly complementarily bind in the sample.

5. The method of claim 4, wherein, in the step (1), a first primer set to a fifth primer set are used to amplify the first region of a target sequence of a strain in the sample; and the second region continuously connected with the first region.

6. The method of claim 5, wherein the first primer set of SEQ ID NO: 16 and SEQ ID NO: 17 amplifies a first region and a second region of a nuc gene of Staphylococcus aureus,
the second primer set of SEQ ID NO: 18 and SEQ ID NO: 19 amplifies a first region and a second region of a mecA gene of methicillin-resistant Staphylococcus aureus,
the third primer set of SEQ ID NO: 20 and SEQ ID NO: 21 amplifies a first region and a second region of a sodA gene of Enterococcus faecium,
the fourth primer set of SEQ ID NO: 22 and SEQ ID NO: 23 amplifies a first region and a second region of a vanA gene of vancomycin-resistant Enterococcus faecium, and
the fifth primer set of SEQ ID NO: 24 and SEQ ID NO: 25 amplifies a first region and a second region of a lytA gene of Streptococcus pneumoniae.

7. The method of claim 4, wherein, in the step (2), the first region of the target sequence of the sample is hybridized to the LHS of the LPO probe and the second region of the target sequence of the sample is hybridized to the RHS of the RPO probe.

8. The method of claim 4, wherein the hybridization in the step (2) is performed for 4 to 20 hours.

9. The method of claim 4, wherein, in the step (3), the end of the LHS of the LPO hybridized to the first region of the sample and the end of the RHS of the RPO hybridized to the second region of the sample are ligated to form the probe assembly composed of the forward primer binding site-LHS-RHS-the reverse primer binding site.

10. The method of claim 4, wherein, in the step (4), a primer set complementarily binding to the forward primer binding site included in the LPO and the reverse primer binding site included in the RPO, which constitute the probe assembly, is used to perform PCR amplification of the probe assembly.

11. The method of claim 4, wherein, in the step (5), an extent at which the probe assembly as a template is PCR-amplified is isolated and detected using capillary electrophoresis-single strand conformation polymorphism (CE-SSCP).

12. The method of claim 4, wherein, in the step (2), the presence of a plurality of gram-positive pathogens is detected at one time using the probe for detecting gram-positive pathogens associated with sepsis according to claim 1.

13. A method of providing information for the diagnosis of sepsis, the method comprising:
(1) a step of performing pre-amplification of a sample;
(2) a step of bringing the pre-amplified sample into contact with the probe for detecting gram-positive pathogens associated with sepsis according to claim 1 to induce hybridization;
(3) a step of ligating the probes hybridized to the sample to form a probe assembly;
(4) a step of performing PCR amplification by using the formed probe assembly as a template;
(5) a step of isolating and detecting the amplified product; and
(6) a step of determining that, when the amplified product is isolated and detected, there are gram-positive pathogens associated with sepsis to which probes constituting the amplified probe assembly complementarily bind in the sample.


SEQUENCE LISTING (25 sequences)

SEQ ID NO: 1 - DNA, 30 bp, Artificial
  Staphylococcus aureus nuc LHS
caacatcacgaaattccttacatgacctcg

SEQ ID NO: 2 - DNA, 29 bp, Artificial
  Staphylococcus aureus nuc RHS
gtttagttcacagaagccgtgttctcatc

SEQ ID NO: 3 - DNA, 35 bp, Artificial
  MRSA mecA LHS
ggtcaataatgtcccagctagaatgcagtatgaaa

SEQ ID NO: 4 - DNA, 28 bp, Artificial
  MRSA mecA RHS
aaataacggctcacagcatggaacaact

SEQ ID NO: 5 - DNA, 33 bp, Artificial
  Enterococcus faecium sodA LHS
cctgcttcgaccttcaaaatgcttaatgctttg

SEQ ID NO: 6 - DNA, 26 bp, Artificial
  Enterococcus faecium sodA RHS
atcggccttgagcaccataaggcaac

SEQ ID NO: 7 - DNA, 27 bp, Artificial
  VREfm vanA LHS
ggctcaggtactgctatccaccctcaa

SEQ ID NO: 8 - DNA, 32 bp, Artificial
  VREfm vanA RHS
acaggtgaattattagcacttgtaagcacacc

SEQ ID NO: 9 - DNA, 33 bp, Artificial
  Streptococcus pneumoniae lytA LHS
catcctaaaaaaggtgtagagaaatatggtcct

SEQ ID NO: 10 - DNA, 29 bp, Artificial
  Streptococcus pneumoniae lytA RHS
gaagcaagtgcatttacgaaaaagatggt

SEQ ID NO: 11 - DNA, 59 bp, Artificial
  Staphylococcus aureus
caacatcacgaaattccttacatgacctcggtttagttcacagaagccgtgttctcatc

SEQ ID NO: 12 - DNA, 63 bp, Artificial
  MRSA
ggtcaataatgtcccagctagaatgcagtatgaaaaaataacggctcacagcatggaacaact

SEQ ID NO: 13 - DNA, 59 bp, Artificial
  Enterococcus faecium
cctgcttcgaccttcaaaatgcttaatgctttgatcggccttgagcaccataaggcaac

SEQ ID NO: 14 - DNA, 59 bp, Artificial
  VREfm
ggctcaggtactgctatccaccctcaaacaggtgaattattagcacttgtaagcacacc

SEQ ID NO: 15 - DNA, 62 bp, Artificial
  Streptococcus pneumoniae
catcctaaaaaaggtgtagagaaatatggtcctgaagcaagtgcatttacgaaaaagatggt

SEQ ID NO: 16 - DNA, 20 bp, Artificial
  Staphylococcus aureus nuc Forward
tacggcaacctctttccatc

SEQ ID NO: 17 - DNA, 20 bp, Artificial
  Staphylococcus aureus nuc Reverse
catgcccttctccctttgta

SEQ ID NO: 18 - DNA, 20 bp, Artificial
  MRSA mecA Forward
agcggtaaacgatttgttgg

SEQ ID NO: 19 - DNA, 20 bp, Artificial
  MRSA mecA Reverse
gatagacttggcgcccataa

SEQ ID NO: 20 - DNA, 20 bp, Artificial
  Enterococcus faecium sodA Forward
agctcagcaaatgcatcaca

SEQ ID NO: 21 - DNA, 20 bp, Artificial
  Enterococcus faecium sodA Reverse
agccaagccttgacgaacta

SEQ ID NO: 22 - DNA, 21 bp, Artificial
  VREfm vanA Forward
ggctatcgtgtcacaatcgtt

SEQ ID NO: 23 - DNA, 20 bp, Artificial
  Synthetic Primer
tggaacttgttgagcagagg

SEQ ID NO: 24 - DNA, 20 bp, Artificial
  Streptococcus pneumoniae lytA Forward
caaatcacagcgcttcaaaa

SEQ ID NO: 25 - DNA, 20 bp, Artificial
  Streptococcus pneumoniae lytA Reverse
aaccaacacgcttcacttcc